(19) United States
(12) Patent Application Publication
(10) Pub. No.: US 2016/0100792 A1
(43) Pub. Date: Apr. 14, 2016

(54) SLEEP STATE DETERMINATION APPARATUS, SLEEP STATE DETERMINATION METHOD, AND SLEEP MANAGEMENT SYSTEM

(71) Applicant: Seiko Epson Corporation, Tokyo (JP)

(72) Inventor: Yimei Ding, Shiojiri-shi (JP)

(21) Appl. No.: 14/847,092

(22) Filed: Sep. 8, 2015

(30) Foreign Application Priority Data
Oct. 8, 2014 (JP) 2014-206972

(51) Int. Cl. A61B 5/00; A61B 5/0205

(52) U.S. Cl. CPC A61B 5/4812; A61B 5/4035; A61B 5/7278; A61B 5/0205; A61B 5/024

(57) ABSTRACT

A sleep state determination apparatus includes: a measurement unit which measures biological information; and a determination unit which calculates a first index in accordance with an activity of a first autonomic nerve and a second index in accordance with an activity of a second autonomic nerve, based on the biological information which is measured by the measurement unit, derives first variation information which indicates a variation of the first index and a second variation information which indicates a variation of the second index, and determines whether a sleep state indicated by the biological information is REM sleep or non-REM sleep, based on the first variation information and the second variation information.

This application claims priority to Japanese Patent Application No. 2014-206972, filed Oct. 8, 2014, the entirety of which is hereby incorporated by reference.

### BACKGROUND

1. Technical Field

The present invention relates to a sleep state determination apparatus, a sleep state determination method, and a sleep management system.

2. Related Art

It is essential to get good quality sleep in order to live healthier life every day, and a demand for analyzing a sleep state has increased as a way of evaluating health conditions.

It has been well known that human sleep is not uniform and is periodic through the night. That is, during human sleep, REM sleep which is accompanied by rapid eye movements and non-REM sleep which is not accompanied by the rapid eye movements periodically occur several times. In a non-REM sleep period, a shallow sleep state is gradually shifted to a deep sleep state which is continued for awhile. Thereafter, the deep sleep state is shifted to the shallow sleep state again, and then, a REM sleep period appears. It has also been known that the above-described period is about 100 minutes (80 minutes to 120 minutes) or the time ratio between the non-REM sleep period and the REM sleep period is about 8:2.

A sleep polygraph device for measuring such sleep states has been known. The sleep polygraph device decodes the stages of sleep through inspection in accordance with the Rechtschaffen & Kales international determination criteria, by installing a number of electrodes in a head portion and a face portion of a subject and simultaneously measuring biological signals such as brain waves, eye movements, and electrical activities in muscles and the heart. Use of such sleep polygraph device is limited to laboratories or hospitals since the device is highly restrictive and expert determination is required, in addition to being expensive.

Therefore, a device which can easily obtain the sleep states at home is expected. For example, in the following JP-A-8-112270, there is a method of determining whether the sleep state is REM sleep or non-REM sleep, from information such as a heart rate and a body motion.

That is, if it is possible to confirm a body motion within a predetermined time, the sleep at that point is determined as non-REM sleep; if it is impossible to confirm a body motion within a predetermined time, a difference value between a current value of a measured heart rate and the lowest value of heart rates until then is calculated; if the difference value is greater than or equal to a predetermined threshold value, it is determined that the sleep state is REM sleep; and if the difference value is less than a predetermined threshold value, it is determined that the sleep state is non-REM sleep.

Although there are individual differences in the heart rate or the body motion, the threshold value for determining REM sleep or non-REM sleep is uniformly determined regardless of individual differences. Therefore, there is a problem in that it is impossible to accurately determine the sleep state. In addition, a body motion is easily generated even when awakening in the middle of sleep. Therefore, there is a problem in that the non-REM sleep and the awakening in the middle of sleep cannot be distinguished from each other.

### SUMMARY

An advantage of some aspects of the invention is to accurately determine a sleep state and an awake state.

The invention can be implemented as the following forms or application examples.

### Application Example 1

A sleep state determination unit according to this application example includes a measurement unit which measures biological information, and a determination unit which calculates a first index in accordance with an activity of a first autonomic nerve and a second index in accordance with an activity of a second autonomic nerve, based on the biological information which is measured by the measurement unit, derives first variation information which indicates a variation of the first index and a second variation information which indicates a variation of the second index, and determines whether asleep state indicated by the biological information is REM sleep or non-REM sleep, based on the first variation information and the second variation information.

According to the configuration, the determination unit calculates the first index in accordance with the activity of the first autonomic nerve and the second index in accordance with the activity of the second autonomic nerve, based on the measured biological information, derives the first variation information and the second variation information which indicate the variation of the calculated first index and the second index respectively, and determines the sleep state based on the derived first variation information and the derived second variation information. Accordingly, it is possible to determine whether the sleep state is REM sleep or non-REM sleep by eliminating the influence in which the biological information varies depending on the individual difference, using the first variation information and the second variation information.

### Application Example 2

In the sleep state determination apparatus according to the application example described above, it is preferable that the determination unit determines whether the sleep state indicated by the biological information is the REM sleep or the non-REM sleep, based on a predetermined area which is defined by the first variation information and the second variation information.

According to the configuration, the determination unit determines the sleep state based on the predetermined area which is defined by the derived first variation information and the derived second variation information. Therefore, it is possible to determine whether the sleep state is REM sleep or non-REM sleep by eliminating the influence in which the biological information varies depending on the individual difference, by appropriately obtaining the predetermined area.

### Application Example 3

In the sleep state determination apparatus according to the application example described above, it is preferable that the biological information is a pulse wave, in which the first index and the second index are calculated by performing frequency analysis on a periodical variation of the pulse wave, and in which the first variation information and the second variation information indicate a variation in accordance with the lapse of time of each of the first index and the second index.

According to the configuration, the first index and the second index are calculated by performing frequency analysis on the periodical variation of the pulse wave, and the variation in accordance with the lapse of time of the first index is obtained as the first variation information and the variation in accordance with the lapse of time of the second index is obtained as the second variation information.

### Application Example 4

In the sleep state determination apparatus according to the application example described above, it is preferable that the predetermined area indicates a time domain in which a first extreme value area in the first variation information and a second extreme value area in the second variation information overlap each other.

According to the configuration, the determination unit determines the sleep state based on the time domain in which the first extreme value area in the first variation information and the second extreme value area in the second variation information overlap each other. Therefore, it is possible to determine whether the sleep state is REM sleep or non-REM sleep, based on whether the predetermined area is included in the time domain in which the first extreme value area and the second extreme value area overlap each other.

### Application Example 5

In the sleep state determination apparatus according to the application example described above, it is preferable that the predetermined area is an area which is defined by a maximum point of the first variation information, a minimum point of the second variation information, and a point at which a value of the first variation information is greater than or equal to a value of the second variation information, in the variation in accordance with the lapse of time of the first variation information and the second variation information.

According to the configuration, the predetermined area can be defined by a maximum point of the first variation information, a minimum point of the second variation information, and a point at which a value of the first variation information is greater than or equal to a value of the second variation information.

### Application Example 6

In the sleep state determination apparatus according to the application example described above, it is preferable that the determination unit determines the sleep state as the REM sleep in a first time zone in which the first extreme value area and the second extreme value area overlap each other, and determines the sleep state as the non-REM sleep in a second time zone in which the first extreme value area and the second extreme value area do not overlap each other.

According to the configuration, it is possible to determine the time zone in which the first extreme value area and the second extreme value area overlap each other as a time zone of REM sleep and to determine the time zone in which the first extreme value area and the second extreme value area do not overlap each other as a time zone of non-REM sleep.

### Application Example 7

In the sleep state determination apparatus according to the application example described above, the determination unit may perform smoothing processing and normalization processing on the first variation information and the second variation information.

### Application Example 8

It is preferable that the sleep state determination apparatus according to the application example described above further includes an age information acquisition unit which acquires age information relating to the age, in which the determination unit determines a first threshold value and a second threshold value based on the age information, determines the first extreme value area by applying the first threshold value to the first variation information, and determines the first extreme value area by applying the second threshold value to the second variation information.

According to the configuration, it is possible to determine the first threshold value and the second threshold value based on the age information and to determine the first extreme value area and the second extreme value area by respectively applying the first threshold value and the second threshold value to the first information and the second information.

### Application Example 9

In the sleep state determination apparatus according to the application example described above, it is preferable that the determination unit determines the sleep state as the REM sleep in a first time zone in which the first extreme value area and the second extreme value area overlap each other, and determines the sleep state as the non-REM sleep in a second time zone in which the first extreme value area and the second extreme value area do not overlap each other.

According to the configuration, it is possible to determine the time zone in which the first extreme value area and the second extreme value area overlap each other as a time zone of REM sleep and to determine the time zone in which the first extreme value area and the second extreme value area do not overlap each other as a time zone of non-REM sleep.

### Application Example 10

In the sleep state determination apparatus according to the application example described above, it is preferable that the determination unit acquires an incidence ratio of the REM sleep in accordance with the age information, determines the first threshold value such that a first proportion which defines the first extreme value area in the first variation information becomes the incidence ratio, and determines the second threshold value such that a second proportion which defines the second extreme value area in the second variation information becomes the incidence ratio.

According to the configuration, the first threshold value and the second threshold value are determined based on the incidence ratio of REM sleep in accordance with the age information, and therefore, it is possible to accurately determine the first extreme value area and the second extreme value area.

### Application Example 11

In the sleep state determination apparatus according to the application example described above, the determination unit divides a power spectrum which is obtained through frequency analysis at predetermined frequencies, one of which is regarded as the first index and the other one of which is regarded as the second index.

According to the configuration, the first index and the second index are determined by performing frequency analysis on the period of the pulse wave and dividing the power spectral density at predetermined frequencies, and therefore, it is possible to correlate the situations of the activities of the autonomic nerves which are synchronized with the period of the pulse wave, to the first index and the second index.

### Application Example 12

In the sleep state determination apparatus according to the application example described above, the measurement unit may detect body motion information, the determination unit may acquire a variation amount indicating a variation of the body motion information and determine whether a subject is in the sleep state or in an awake state, based on the acquired variation amount.

According to the configuration, it is possible to determine whether a subject is in the sleep state or in the awake state, based on the variation amount indicating the variation of the body motion information.

### Application Example 13

In the sleep state determination apparatus according to the application example, the determination unit may calculate a first variance between a first class and a second class, and a second variance within each of the classes, and determines a threshold value, for classifying the variation amount into the first class for determining the awake state and the second class for determining the sleep state, such that the ratio of the first variance and the second variance becomes maximum.

### Application Example 14

A sleep state determination method according to this application example includes measuring biological information; calculating a first index in accordance with an activity of a first autonomic nerve and a second index in accordance with an activity of a second autonomic nerve, based on the measured biological information, deriving first variation information which indicates a variation of the first index and a second variation information which indicates a variation of the second index, and determining whether a sleep state indicated by the biological information is REM sleep or non-REM sleep, based on the first variation information and the second variation information.

According to the method, the first index is calculated in accordance with the activity of the first autonomic nerve and the second index is calculated in accordance with the activity of the second autonomic nerve, based on the measured biological information; the first variation information and the second variation information which indicate the variations of the calculated first index and the calculated second index are derived; and the sleep state is determined based on the derived first variation information and the derived second variation information. Accordingly, it is possible to determine whether the sleep state is REM sleep or non-REM sleep by eliminating the influence in which the biological information varies depending on the individual difference, using the first variation information and the second variation information.

### Application Example 15

A sleep management system according to this application example is a sleep management system in which a sleep state determination apparatus that determines a sleep state and an information processing apparatus are communicably connected to each other, in which the sleep state determination apparatus includes a measurement unit which measures biological information, a determination unit which calculates a first index in accordance with an activity of a first autonomic nerve and a second index in accordance with an activity of a second autonomic nerve, based on the biological information which is measured by the measurement unit, derives first variation information which indicates a variation of the first index and second variation information which indicates a variation of the second index, and determines whether asleep state indicated by the biological information is a REM sleep or a non-REM sleep, based on the first variation information and the second variation information, and a communication unit which notifies the information processing apparatus of a determination result of the determination unit through communication, and in which the information processing apparatus processes information relating to sleep based on the determination result which is notified from the sleep state determination apparatus.

According to the configuration, the first index in accordance with the activity of the first autonomic nerve and the second index in accordance with the activity of the second autonomic nerve are calculated based on the measured biological information; the first variation information and the second variation information which indicate the variations of the calculated first index and the calculated second index are derived; and the sleep state is determined based on the derived first variation information and the derived second variation information. Accordingly, it is possible to determine whether the sleep state is REM sleep or non-REM sleep by eliminating the influence in which the biological information varies depending on the individual difference, using the first variation information and the second variation information, and to perform information processing on the determination result in the information processing apparatus.

### BRIEF DESCRIPTION OF THE DRAWINGS

The invention will be described with reference to the accompanying drawings, wherein like numbers reference like elements.

FIG. 1 is a view showing a functional configuration of a sleep management system according to Embodiment 1 of the invention.

FIG. 2 is a view illustrating signal processing.

FIG. 3 is a view showing an example of inputting signals into a REM sleep area determination unit.

FIG. 4 is a view showing a hardware configuration of a sleep state determination apparatus.

FIG. 5 is a flowchart showing a flow of processing of determining a sleep state.

FIG. 6 is a flowchart showing a flow of processing of determining a REM sleep area in Embodiment 1.

FIG. 7 is a view showing a functional configuration of a sleep management system according to Embodiment 2.

FIG. 8 is a view showing an occurrence rate of REM sleep in accordance with age.

FIG. 9 is a flowchart showing a flow of processing of determining a REM sleep area in Embodiment 2.

### DESCRIPTION OF EXEMPLARY EMBODIMENTS

Hereinafter, embodiments of the invention will be described with reference to the drawings.

### Embodiment 1

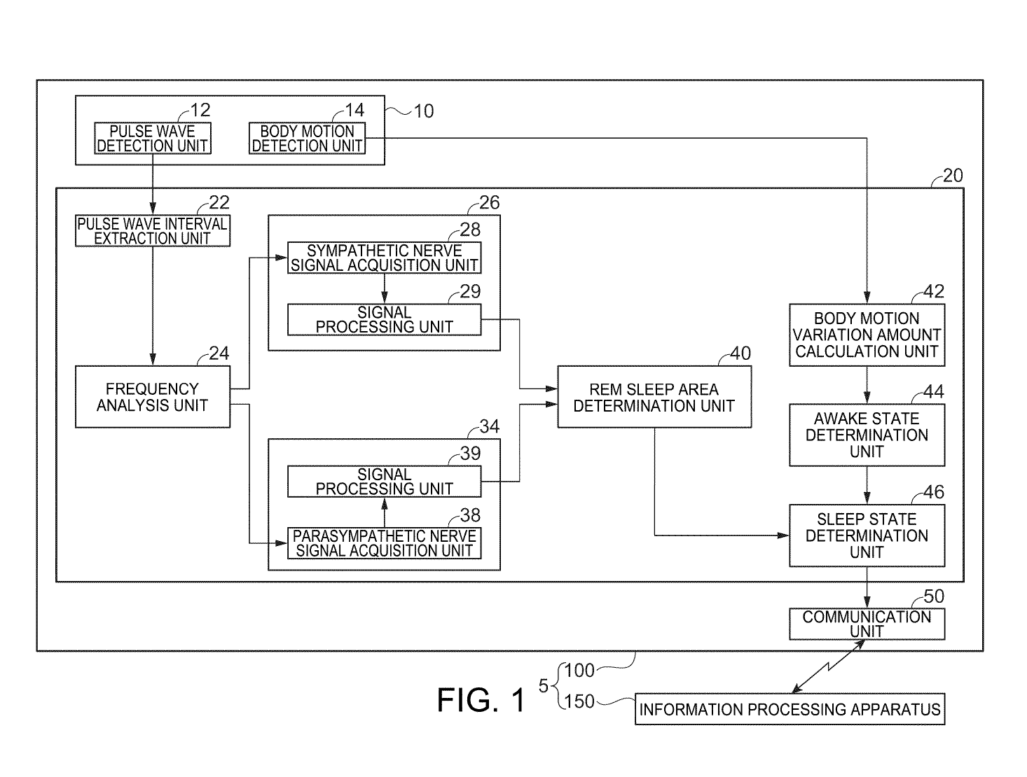

FIG. 1 is a view showing a functional configuration of a sleep management system 5 according to the present Embodiment 1. The sleep management system 5 includes a sleep state determination apparatus 100 which determines a sleep state and an information processing apparatus 150 which manages the sleep state. The sleep state determination apparatus 100 and the information processing apparatus 150 are communicably connected to each other through short-distance radio or the like.

In the present Embodiment 1, a mode is assumed in which the sleep state determination apparatus 100, such as a wristwatch, is worn on the body of a subject, for example, on an arm portion, and the information processing apparatus 150 is assumed to be a highly functional portable telephone such as a smart phone or a multifunctional mobile terminal such as a tablet terminal. However, the invention is not limited thereto.

### Configuration of Sleep State Determination Apparatus

The sleep state determination apparatus 100 includes a measurement unit 10 which measures biological information; a processing unit 20 which processes a biological information signal which is measured by the measurement unit 10; and a communication unit 50 which communicates with the information processing apparatus 150. The processing unit 20 corresponds to a determination unit.

The measurement unit 10 includes a pulse wave detection unit 12 which detects a pulse of a subject and outputs a pulse wave signal as one biological information signal; and a body motion detection unit 14 which detects a body motion of a subject and outputs a body motion signal as one biological information signal.

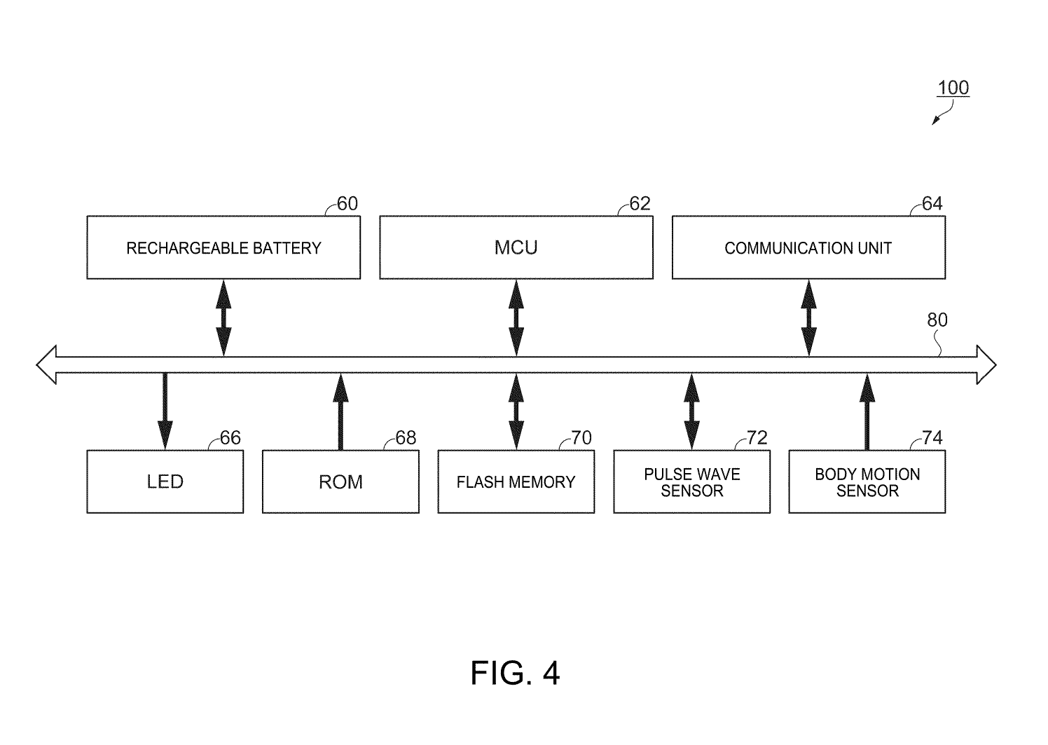

In the present Embodiment 1, the pulse wave detection unit 12 includes a pulse wave sensor 72 (FIG. 4) which optically detects a pulse wave and generates a pulse wave signal in accordance with the detected pulse wave. When the sleep state determination apparatus 100 is worn on an arm portion of a subject, the pulse wave sensor 72 is disposed so as to be in close contact with and face the arm portion of the subject. The pulse wave detection unit 12 outputs the generated pulse wave signal to the processing unit 20.

In addition, the body motion detection unit 14 includes a body motion sensor 74 (FIG. 4). The body motion sensor 74 generates an acceleration accompanied by a movement of the body of the detected subject as a body motion signal (body motion information). The body motion detection unit 14 outputs the generated body motion signal to the processing unit 20.

The processing unit 20 includes a pulse wave interval extraction unit 22, a frequency analysis unit 24, a sympathetic nerve signal processing unit 26, a parasympathetic nerve signal processing unit 34, a REM sleep area determination unit 40, a body motion variation amount calculation unit 42, an awake state determination unit 44, and a sleep state determination unit 46.

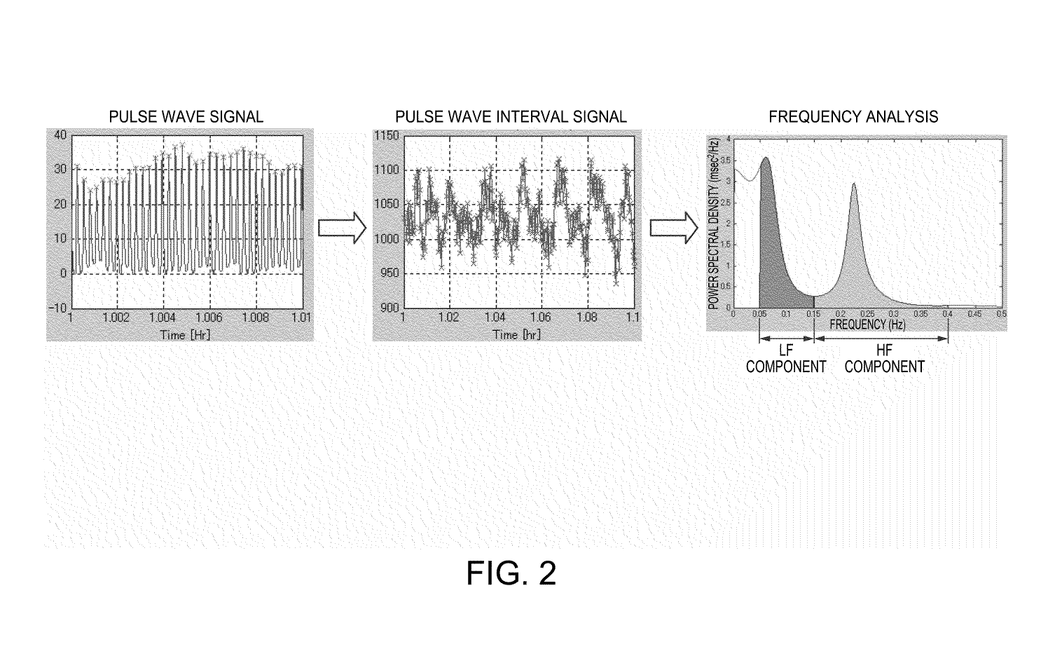

The pulse wave interval extraction unit 22 acquires a pulse wave signal which is output from the pulse wave detection unit 12 through sleep and extracts a pulse wave interval of the obtained pulse wave signal. In the present Embodiment 1, as shown in FIG. 2 which illustrates signal processing, the pulse wave interval extraction unit 22 extracts extreme values of pulse wave signals which change in accordance with the lapse of time and outputs the extracted extreme values as pulse wave interval signals showing a time interval between the extracted extreme values, that is, a period of pulse waves, to the frequency analysis unit 24. Maximum values of the pulse wave signals were extracted in the present Embodiment 1. However, the invention is not limited thereto, and minimum values may be extracted or the period of pulse waves may be acquired from an interval of a point (zero-cross point) at which polarity of the pulse waves change.

As is well known, the pulse waves indicated by the pulse wave signals indicate blood flow change of a blood vessel of a subject and vary in synchronization with a heartbeat. Accordingly, it is possible to capture a pulse wave interval indicated by a pulse wave interval signal as a heartbeat interval.

The frequency analysis unit 24 performs frequency analysis based on the pulse wave interval signal which is output from the pulse wave interval extraction unit 22. In the present Embodiment 1, as shown in FIG. 2, the frequency analysis unit 24 performs frequency analysis on the pulse wave interval signal, that is, on a periodical variation in pulse waves, and outputs distribution information of power spectral density (may also be simply referred to as a power spectrum) in a frequency band to the sympathetic nerve signal processing unit 26 and the parasympathetic nerve signal processing unit 34 which process a signal relating to an autonomic nerve.

As is well known, in the distribution of the power spectral density which is obtained by analyzing the frequency of the pulse wave interval signal which is synchronized with the heartbeat interval, one low frequency band (LF component: 0.05 Hz to 0.15 Hz) which is divided at predetermined frequencies indicates an active state of a sympathetic nerve (first autonomic nerve) which is one autonomic nerve, and another high frequency band (HF component: 0.15 Hz to 0.4 Hz) indicates an active state of a parasympathetic nerve (second autonomic nerve) which is the other autonomic nerve. In addition, it has been known that there is a certain correlation between REM sleep and non-REM sleep, and the active states of the autonomic nerves. Accordingly, it is possible to estimate whether a subject is in a REM sleep state or in a non-REM sleep state, by analyzing the LF component and the HF component as an autonomic nerve index.

The frequency band of the pulse wave interval signal is analyzed in the present Embodiment 1. However, the invention is not limited thereto and a mode of analyzing a time band can also be assumed.

The sympathetic nerve signal processing unit 26 includes a sympathetic nerve signal acquisition unit 28 and a signal processing unit 29. In addition, the parasympathetic nerve signal processing unit 34 includes a parasympathetic nerve signal acquisition unit 38 and a signal processing unit 39.

The sympathetic nerve signal acquisition unit 28 acquires a ratio (first index: LF/HF component) of a power value, which indicates the intensities of the LF component and the HF component, at a predetermined time interval from distribution information of the power spectral density, and sends a signal of the acquired LF/HF component to the signal processing unit 29 as an index of sympathetic nerve activity.

The signal processing unit 29 performs smoothing processing on the signal of the LF/HF component, and then, performs normalization processing on the signal of the LF/HF component such that the signals are within, for example, the range of 0 to 1, and sends the normalized signals of the LF/HF component to the REM sleep area determination unit 40.

The parasympathetic nerve signal acquisition unit 38 acquires a power value of an HF component (second index) from the distribution information of the power spectral density at a predetermined time interval, and sends the acquired signal of the HF component to the signal processing unit 39 as an index of parasympathetic nerve activity.

Similarly to the signal processing unit 29, the signal processing unit 39 performs smoothing processing on the signal of the HF component, and then, performs normalization processing on the signal of the HF component and sends the normalized signal of the HF component to the REM sleep area determination unit 40.

The REM sleep area determination unit 40 determines an area which is determined as REM sleep, based on the distribution of a predetermined area which is defined by the signal of the LF/HF component and the signal of the HF component which are obtained from the sympathetic nerve signal processing unit 26 and the parasympathetic nerve signal processing unit 34.

In general, it is known that the HF component tends to be low and the LF/HF component tends to be high in a REM sleep state. The REM sleep area determination unit 40 determines the sleep state by detecting the tendency.

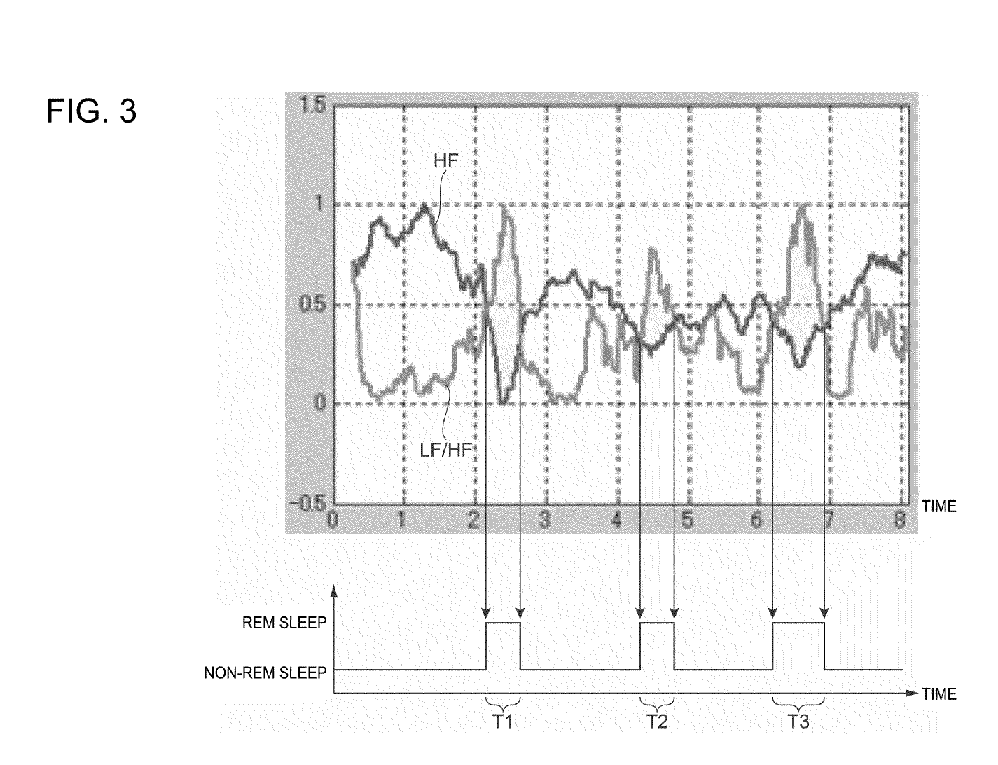

That is, in the present Embodiment 1, as shown in the example of inputting signals into the REM sleep area determination unit 40 which is shown in FIG. 3, the REM sleep area determination unit 40 develops variations in the input signal of the LF/HF component and the signal of the HF component in accordance with the lapse of time, on an identical time axis.

Next, the REM sleep area determination unit 40 determines first time zones (T1, T2, and T3), in which when the time at which the LF/HF component and the HF component intersect with each other are regarded as a start point or an end point, a peak area (first extreme value area) that includes a maximum value (maximum point) in first variation information indicating a time variation in the LF/HF component and a peak area (second extreme value area) that includes a minimum value (minimum point) in the second variation information indicating a time variation in the HF component overlap each other, and a value of the first variation information is greater than or equal to a value of the second variation information, as REM sleep areas, and determines other second time zones, which do not overlap with the first time zones, as non-REM sleep areas.

In the above-described method, since the areas are determined based on the overlap of the peak area of the LF/HF component and the peak area of the HF component which are normalized and derived from a measured pulse wave signal, it is unnecessary to separately set a threshold value which can vary depending on the individual difference between subjects.

The REM sleep area determination unit 40 sends information of the sleep areas which are determined in this manner to the sleep state determination unit 46.

The body motion variation amount calculation unit 42 acquires a body motion signal which is output from the body motion detection unit 14 and calculates the number of body motions of a subject based on the acquired body motion sensor. For example, the body motion variation amount calculation unit 42 performs arithmetic processing on an acceleration vector in 3 axis directions which is output from the body motion sensor 74 and calculates the number of body motions of the subject using an acceleration value from which a DC component is removed. The technique of obtaining the number of body motions from an output from a sensor which detects acceleration is disclosed in, for example, JP-A-2004-89267.

Furthermore, the body motion variation amount calculation unit 42 performs statistical processing on the number of body motions which changes in accordance with the lapse of time and calculates the variation amount of the number of body motions. The body motion variation amount calculation unit 42 sends the information of the calculated variation amount of the number of body motions to the awake state determination unit 44.

The awake state determination unit 44 determines whether the subject is in a sleep state or in an awake state based on the information of the variation amount of the number of body motions which is output from the body motion variation amount calculation unit 42.

In the present Embodiment 1, the awake state determination unit 44 develops the variation amount of the number of body motions on a time axis and determines a threshold value for determining the sleep state or the awake state.

Here, for example, a method through discrimination analysis which is known as “Otsu's method” is employed for the determination of the threshold value. In this method, when classifying a group consisting of a plurality of measurement values into a plurality of groups, the group is classified by setting a threshold value based on the measurement values without using a predetermined threshold value, and therefore, it is possible to perform the classification based on the measurement values. Accordingly, this method can set the threshold value in accordance with the measurement values compared to the technique of uniquely classifying the group using a predetermined threshold value, and therefore, is an effective method for classification of measurement values at which the threshold value tends to vary like the case of determining awake/sleep. The awake state determination unit 44 classifies the variation amount of the number of body motions into a first class for temporarily determining an awake state and a second class for temporarily determining a sleep state, and determines an optimum value of the classified threshold value (degree of separation).

That is, the awake state determination unit 44 calculates variance (first variance) between the classes including the first class and the second class, and variance (second variance) in each of the classes, and determines a threshold value at which the ratio of the second variance to the first variance (first variance/second variance) becomes maximum. The method of determining a threshold value is not limited to the “Otsu's method”. For example, the method may be constituted such that a predetermined threshold value is set in advance and the sleep/awake state is determined based on the setting. For example, the method may be constituted such that a predetermined threshold value is set with respect to the number of body motions or the body motion signal, and the awake state and the sleep state are determined in comparison with the threshold value, or the sleep state may be determined based on the pulse rate.

The awake state determination unit 44 determines a threshold value through the above-described method and determines whether a subject is in a sleep state or in an awake state in a predetermined time based on the determined threshold value. The awake state determination unit 44 sends awake state information, which is obtained as a result of determination over a time zone that is to be focused on, to the sleep state determination unit 46.

The sleep state determination unit 46 determines the sleep state of a subject in a time zone which is to be focused on, that is, any of states including an awake state, a REM sleep state, and a non-REM sleep state, based on the awake state information which is sent from the awake state determination unit 44 and the information of the sleep area which is sent from the REM sleep area determination unit 40, and sends determination results indicating the information of the sleep state from the communication unit 50 to the information processing apparatus 150.

FIG. 4 is a view showing a hardware configuration of the sleep state determination apparatus 100. The hardware of the sleep state determination apparatus 100 is constituted of a rechargeable battery 60, a micro control unit (MCU) 62, a communication unit 64, a light emitting diode (LED) 66, a read only memory (ROM) 68, a flash memory 70, a pulse wave sensor 72, a body motion sensor 74, and the like, which are connected to each other through a bus 80.

The body motion sensor 74 is assumed to be an acceleration sensor or a gyro sensor, and the LED 66 is assumed to be in a mode of functioning as a notification unit which notifies a subject of a measurement operation or generation of an error using a lighting pattern or the like. In addition, the flash memory 70 temporarily stores data output from the pulse wave sensor 72 or the body motion sensor 74 or each data piece which is calculated by each of the above-described functional units and varies in accordance with the lapse of time.

Each functional unit of the processing unit 20 realizes each function through cooperation between the hardware and software which is stored in the ROM 68 and the flash memory 70.

The information processing apparatus 150 receives information of the sleep state which is sent from the sleep state determination apparatus 100, and displays the transition of the sleep state of a subject or displays awake time, REM sleep time, and non-REM sleep time with numerical values or a graph, depending on a request operation of the subject. In addition, a mode can also be assumed in which the received information pieces of the sleep state are accumulated, the transition of the sleep state for each individual is statistically processed to be utilized as an index of health management and to be applied for the improvement of lifestyle. In addition, a mode can also be assumed in which the information processing apparatus 150 transmits an operation instruction signal which instructs collection and transmission of the information of the sleep state, to the sleep state determination apparatus 100.

### Determination Processing Using Sleep State Determination Apparatus

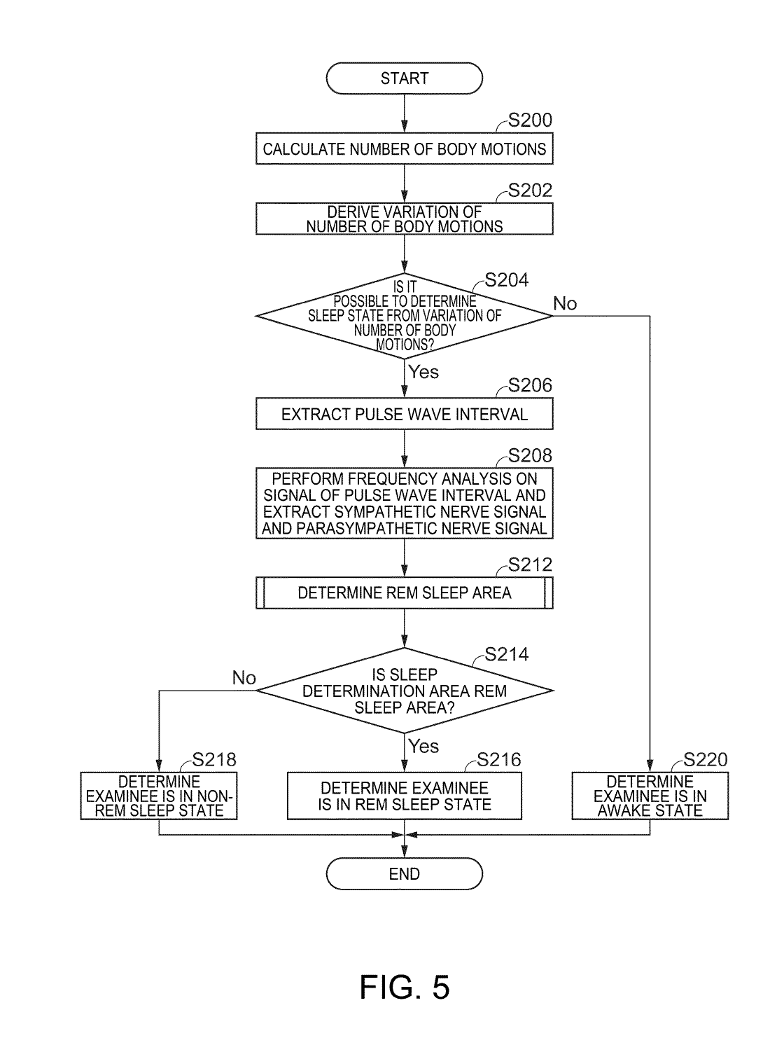

Next, processing of determining a sleep state will be described. FIG. 5 is a flowchart showing a flow of processing of a sleep state determination method in which the sleep state determination apparatus 100 determines the sleep state.

When the processing starts, the processing unit 20 measures biological information of a subject <measurement step>, calculates the number of body motions from body motion signals of the measured biological information (step S200), and derives a variation in the calculated number of body motions (step S202).

Next, the processing unit 20 determines whether a subject is in a sleep state in a sleep determination area that determines a sleep state (step S204), from the variation of the number of body motions. When it is impossible to determine that the subject is in a sleep state (No in step S204), the processing unit 20 determines that the subject is in an awake state (step S220), and the processing is completed.

In contrast, when it is possible to determine that the subject is in a sleep state (Yes in step S204), the processing unit 20 extracts a signal of a pulse wave interval from a pulse wave signal which is one of biological information pieces of the subject (step S206)<calculation step>.

Next, the processing unit 20 performs frequency analysis on the extracted signal of the pulse wave interval and extracts a sympathetic nerve signal and a parasympathetic nerve signal (step S208)<derivation step>.

Next, the processing unit 20 performs processing (step S212) of determining a REM sleep area.

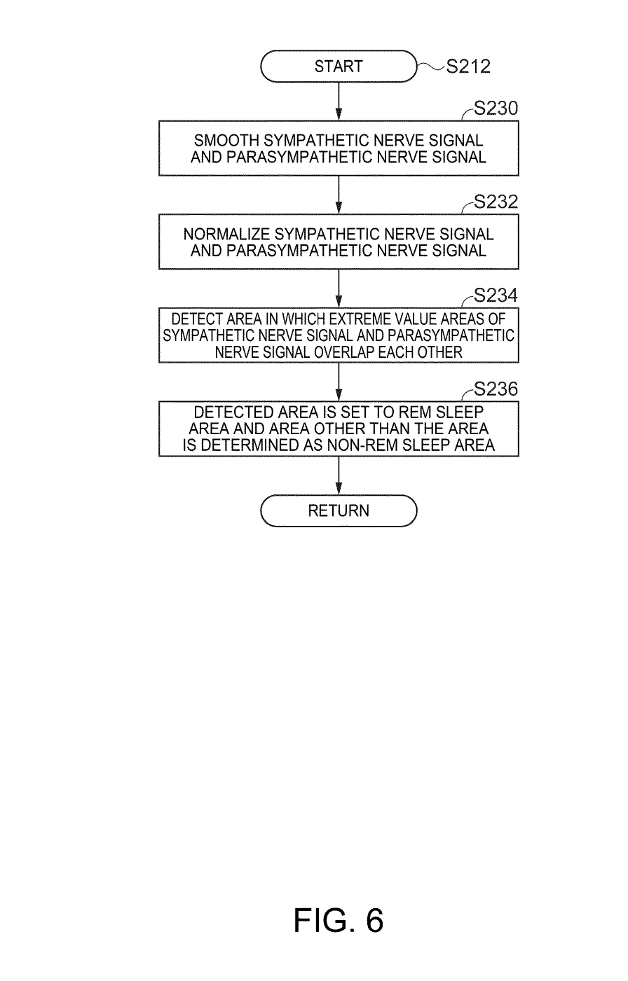

FIG. 6 is a flowchart showing a flow of processing (step S212) of determining a REM sleep area, and this processing will be described below.

When this processing is performed, the processing unit 20 smoothes the extracted sympathetic nerve signal and parasympathetic nerve signal (step S230) and further normalizes the smoothed sympathetic nerve signal and parasympathetic nerve signal (step S232).

Next, the processing unit 20 detects a time domain in which areas including extreme values of the smoothed sympathetic nerve signal and parasympathetic nerve signal overlap each other (step S234).

Next, the processing unit 20 sets the overlapping time domain as a REM sleep area and an area other than the area is determined as a non-REM sleep area (step S236), and the process returns to the step S212.

Returning to FIG. 5, the processing unit 20 determines whether the sleep determination area which determines the sleep state is the REM sleep area (step S214) <determination step>.

Here, when the sleep determination area is a REM sleep area (Yes in step S214), it is determined that a subject is in a REM sleep state (step S216) and the processing is completed.

In contrast, when the sleep determination area is not a REM sleep area (No in step S214), it is determined that a subject is in a non-REM sleep state (step S218) and the processing is completed.

According to the above-described Embodiment 1, the following effect is exhibited.

(1) The frequency of a pulse wave interval signal is analyzed; the ratio (LF/HF component) of a power value of an LF component and an HF component which are acquired from distribution information of power spectral density is regarded as an index of sympathetic nerve activity; a signal of the HF component is regarded as an index of parasympathetic nerve activity; and the REM sleep area and the non-REM sleep area are determined through detection of a time domain in which areas of the two indexes which are smoothed and normalized overlap each other. Accordingly, it is unnecessary to set a threshold value for determining the sleep state which varies depending on the physical condition of a subject or the individual difference, and it is possible to avoid erroneous determination due to the physical condition or the individual difference.

(2) A threshold value which determines any of an awake state and a sleep state using the “Otsu's method” by calculating the variation amount of body motions of a subject is determined, and therefore, it is possible to accurately determine whether the subject is in an awake state or in a sleep state. Moreover, it is possible to avoid erroneous determination as REM sleep or non-REM sleep by detecting awakening even when the subject is awaked in the middle of sleep since it is not determined as a REM sleep area nor a non-REM sleep area when the subject is in an awake state.

### Embodiment 2

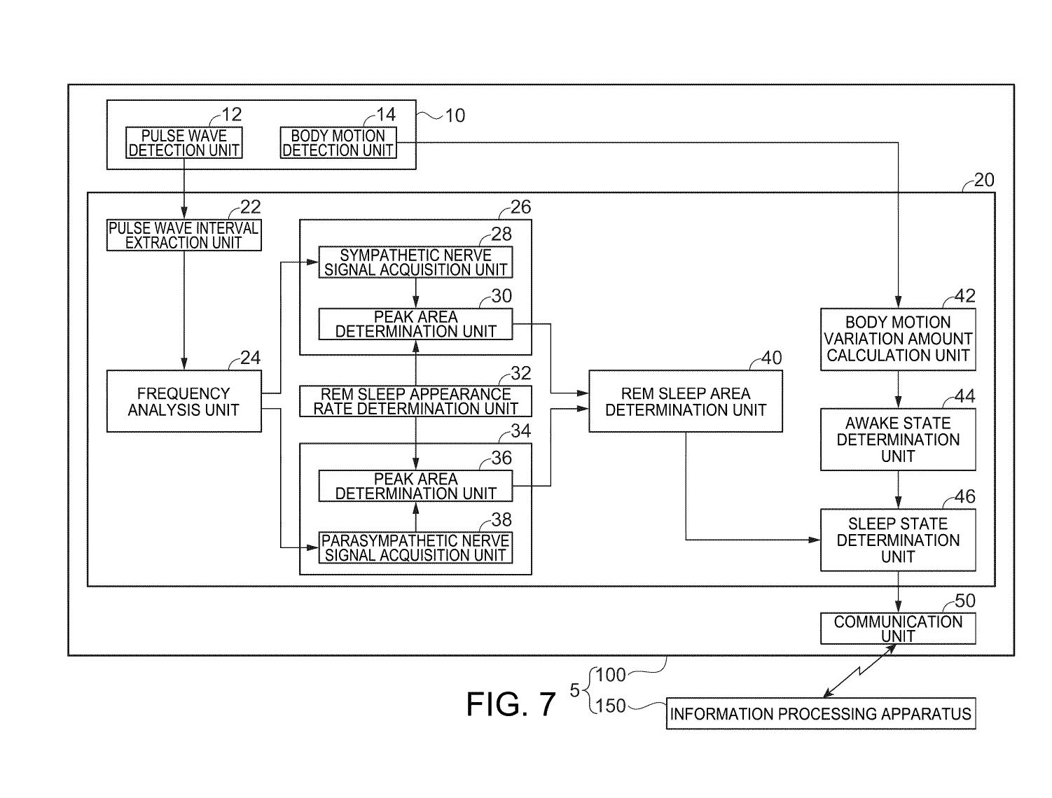

Next, Embodiment 2 of the invention will be described with reference to FIGS. 7 to 9. In the following description, a portion, which has already been described, and the same portion are given the same reference numerals and the description thereof will not be repeated.

FIG. 7 is a view showing a functional configuration of a sleep management system 5 according to Embodiment 2. In the present Embodiment 2, the processing unit 20 further includes a REM sleep occurrence rate determination unit 32. In addition, the sympathetic nerve signal processing unit 26 includes a peak area determination unit 30 instead of the signal processing unit 29 in Embodiment 1 and the parasympathetic nerve signal processing unit 34 includes a peak area determination unit 36 instead of the signal processing unit 39 in Embodiment 1.

The sympathetic nerve signal acquisition unit 28 acquires a ratio (LF/HF component) of a power value of an LF component and an HF component, which are acquired from distribution information of a power spectrum, at a predetermined time interval, and sends a signal of the acquired LF/HF component to the peak area determination unit 30 as an index of sympathetic nerve activity.

In addition, the parasympathetic nerve signal acquisition unit 38 acquires a power value of a HF component from the distribution information of the power spectrum at a predetermined time interval, and sends the acquired signal of the HF component to the peak area determination unit 36 as an index of parasympathetic nerve activity.

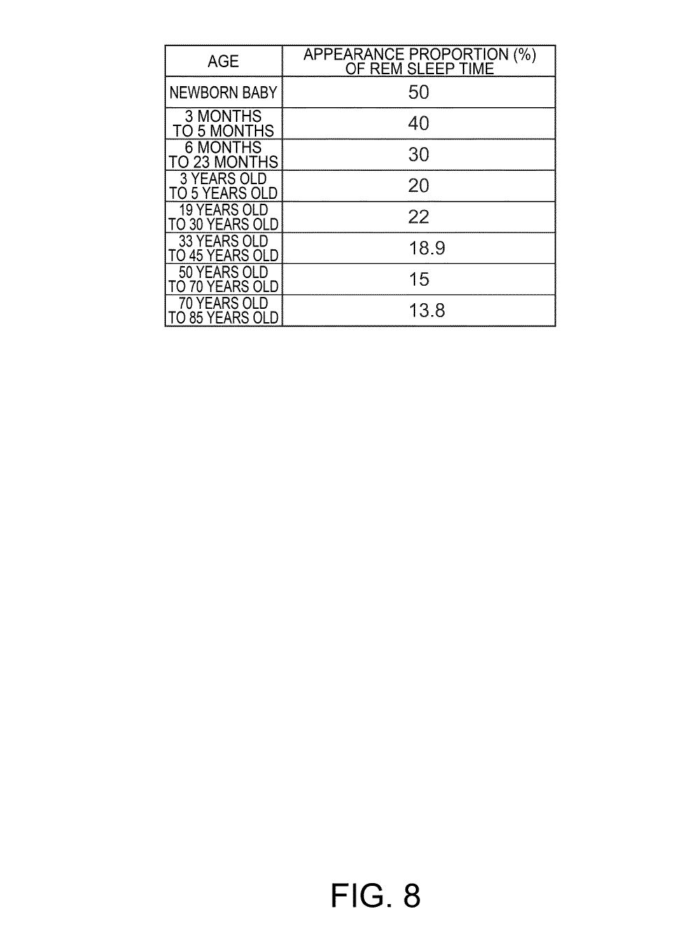

The REM sleep occurrence rate determination unit 32 determines an occurrence rate (incidence ratio) of REM sleep. As is well known, the occurrence rate at which REM sleep appears during sleep changes in accordance with the age. For example, it has been known that REM sleep appears at incidence ratios as shown in FIG. 8. Accordingly, the REM sleep occurrence rate determination unit 32 has the table values as shown in FIG. 8 and determines an occurrence rate of REM sleep based on the age information of a subject which is input in advance.

The REM sleep occurrence rate determination unit 32 sends information of the determined occurrence rate of REM sleep to the peak area determination unit 30 and the peak area determination unit 36. The age information acquisition function in the REM sleep occurrence rate determination unit 32 corresponds to an age information acquisition unit.

The peak area determination unit 30 and the peak area determination unit 36 determine a peak area which defines REM sleep based on the information of the occurrence rate of REM sleep which is acquired from the age information.

The peak area determination unit 30 first acquires a signal of LF/HF component and develops the signal on a time axis, and calculates a frequency distribution. The direction in which a peak is generated in the LF/HF component is a direction in which the values of the LF/HF component become large.

Subsequently, the peak area determination unit 30 calculates an incidence ratio (first proportion) at each bin by sequentially integrating frequencies from a maximum value to a low value of the LF/HF component, and obtains a bin at which the incidence ratio becomes substantially the same as the occurrence rate of REM sleep which is determined by the REM sleep occurrence rate determination unit 32. The peak area determination unit 30 determines a first threshold value which defines a peak area of the LF/HF component at a central position of the bin.

The peak area determination unit 30 cuts a signal of the LF/HF component which is obtained through sleep by applying the determined first threshold value, and sends the information of the peak area of the cut LF/HF component to the REM sleep area determination unit 40.

In addition, the peak area determination unit 30 first acquires a signal of an HF component and develops the signal on a time axis, and calculates a distribution of a power spectrum. The direction in which a peak is generated in the HF component is a direction in which the value of the HF component becomes small.

Consequently, the peak area determination unit 30 calculates an incidence ratio (second proportion) at each bin by sequentially integrating frequencies from a minimum value to a high value of the HF component, and obtains a bin at which the incidence ratio becomes substantially the same as the occurrence rate of REM sleep which is determined by the REM sleep occurrence rate determination unit 32. The peak area determination unit 30 determines a second threshold value which defines a peak area of the HF component at a central position of the bin.

The peak area determination unit 36 cuts a signal of the HF component which is obtained through sleep by applying the determined second threshold value, and sends the information of the peak area of the cut HF component to the REM sleep area determination unit 40.

The REM sleep area determination unit 40 develops the information of the peak area of the LF/HF component which is sent from the peak area determination unit 30 and the information of the peak area of the HF component which is sent from the peak area determination unit 36 on an identical time axis; determines a first time zone, in which a time zone of the peak area of the LF/HF component and a time zone of the peak area of the HF component overlap each other, as a REM sleep area; and determines a second time zone, in which the time zones do not overlap each other, as a non-REM sleep area.

For example, in a case where one peak area of the LF/HF component is 2 to 3 hours after sleep and one peak area of the HF component is 2 hours and 15 minutes to 3 hours and 30 minutes after sleep, the REM sleep area determination unit 40 determines the first time zone as 2 hours and 15 minutes to 3 hours after sleep.

The REM sleep area determination unit 40 sends the information of the sleep area which has been determined in this manner to the sleep state determination unit 46.

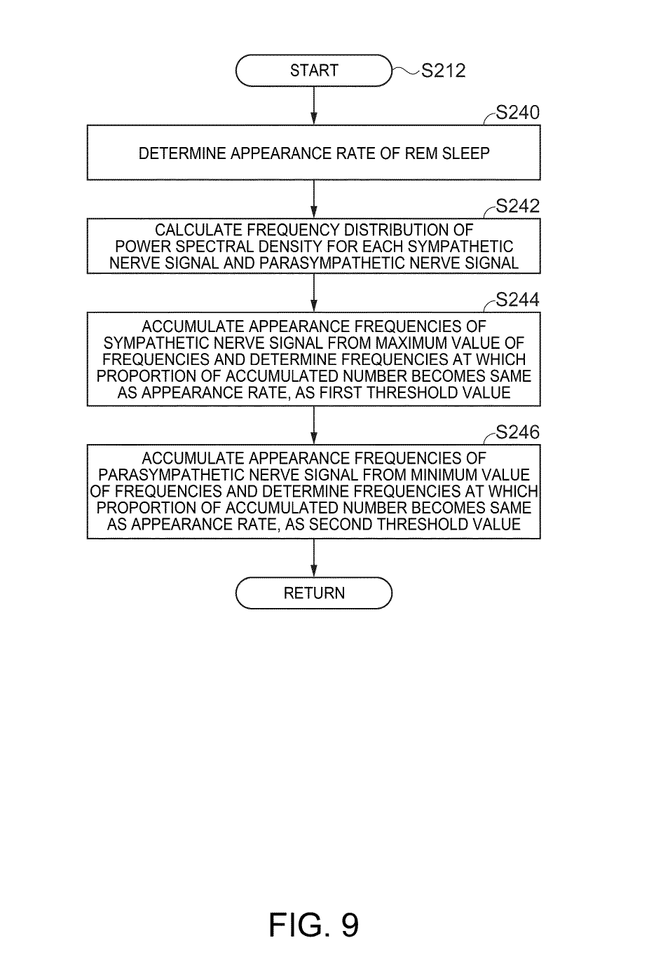

FIG. 9 is a flowchart showing processing (step S212) of determining a REM sleep area in the present Embodiment 2, and this processing will be described below.

First, the processing unit 20 determines an occurrence rate of REM sleep based on age information of a subject (step S240).

Next, the processing unit 20 calculates a frequency distribution for each LF/HF component which is a sympathetic nerve signal and for each HF component which is a parasympathetic nerve signal (step S242).

Next, the processing unit 20 accumulates frequencies from a maximum value of the LF/HF component and determines a central position of a bin, at which the proportion of accumulated number in the total number becomes substantially the same as the occurrence rate of REM sleep, as a first threshold value (step S244).

Next, the processing unit 20 accumulates frequencies from a minimum value of the HF component and determines a central position of a bin, at which the proportion of accumulated number in the total number becomes substantially the same as the occurrence rate of REM sleep, as a second threshold value (step S246), and the process returns to step S212.

According to the above-described Embodiment 2, the following effect is exhibited in addition to the effect of (2) which is described in Embodiment 1.

(3) The frequency of a pulse wave interval signal is analyzed; the ratio (LF/HF component) of a power value of an LF component and an HF component which are acquired from distribution information of power spectral density is regarded as an index of sympathetic nerve activity; a signal of the HF component is regarded as an index of parasympathetic nerve activity; and a threshold value is determined such that the proportion of accumulated number of each frequency distribution and the occurrence rate of REM sleep which has been determined based on the age become substantially the same as each other. Accordingly, it is unnecessary to perform smoothing processing or normalization processing, and it is possible to promptly and easily determine the sleep state of a subject.

The embodiments of the invention have been described with reference to the drawings. However, the specific configuration is not limited to the embodiments and includes modifications in design within a range not departing from the gist of the invention. For example, a mode can also be assumed in which the sleep state determination apparatus 100 is operated independently from the information processing apparatus 150. For example, a mode can also be assumed in which the sleep state determination apparatus 100 includes a display unit for displaying information or an operation unit for inputting an operating instruction and a subject who is worn the sleep state determination apparatus 100 operates the operation unit, to collect information of the sleep state and display the collected information of the sleep state.

Furthermore, a mode can also be assumed in which the sleep state determination apparatus 100 stores information of sleep states which have been collected previously and estimates a current sleep state of a subject based on the stored information of the sleep states.

In addition, in Embodiment 2, the invention is not limited to be in a mode in which the age information of a subject for determining the occurrence rate of REM sleep is previously input, and the age may be estimated based on the pulse rate or the number of body motions of a subject. In addition, a mode can be assumed in which the threshold value for cutting peak areas of the LF/HF component and the HF component is modified through a learning function by the sleep state determination apparatus 100 being repeatedly used by a subject.

Each of the configurations and the combination thereof in each of the embodiments are merely examples, and addition, omission, replacement, and other modifications of the configurations can be made within the scope not departing from the gist of the invention. In addition, the invention is not limited by the embodiments, but is limited by the scope of the appended claims.

In Embodiments 1 and 2, the invention has a configuration in which the sleep state is determined based on the pulse wave signal from the pulse wave detection unit 12, but is not limited thereto. For example, the invention may have a configuration in which an electrocardiograph is used as an electrocardiographic detection unit instead of the pulse wave detection unit 12 or may have a configuration in which both of the pulse wave detection unit and the electrocardiographic detection unit may be used. With such a configuration, for example, the determination of awake/sleep is performed based on the signal from the pulse wave detection unit 12, and when it is determined that a subject is in a sleep state, it is possible to perform determination of the depth of sleep based on the signal from the electrocardiographic detection unit, that is, determination of discriminating between REM sleep/non-REM sleep. Accordingly, it is possible to more accurately discriminate the sleep state.

In Embodiment 1, the invention has a configuration in which the sleep state (sleep/awake state) is discriminated by classifying a group consisting of a plurality of measurement values of a pulse wave interval into a plurality of groups using a threshold value which is set based on the plurality of measurement values of the pulse wave interval. The invention may have a configuration including a presentation unit in which information relating to change in body rhythm of a user or the quality of sleep is presented to the user, by recording the threshold value over a predetermined period, for example, for one year and analyzing the change in the threshold value over time. With such a configuration, it is possible to grasp the state of a user's own body and to increase motivation of continuously performing the measurement.

What is claimed is:

1. A sleep state determination apparatus comprising:
a measurement unit which measures biological information; and
a determination unit which calculates a first index in accordance with an activity of a first autonomic nerve and a second index in accordance with an activity of a second autonomic nerve, based on the biological information which is measured by the measurement unit, derives first variation information which indicates a variation of the first index and a second variation information which indicates a variation of the second index, and determines whether a sleep state indicated by the biological information is REM sleep or non-REM sleep, based on the first variation information and the second variation information.

2. The sleep state determination apparatus according to claim 1,
wherein the determination unit determines whether the sleep state indicated by the biological information is the REM sleep or the non-REM sleep, based on a predetermined area which is defined by the first variation information and the second variation information.

3. The sleep state determination apparatus according to claim 1,
wherein the biological information is a pulse wave,
wherein the first index and the second index are calculated by performing frequency analysis on a periodical variation of the pulse wave, and
wherein the first variation information and the second variation information indicate a variation in accordance with the lapse of time of each of the first index and the second index.

4. The sleep state determination apparatus according to claim 1,
wherein the predetermined area indicates a time domain in which a first extreme value area in the first variation information and a second extreme value area in the second variation information overlap each other.

5. The sleep state determination apparatus according to claim 4,
wherein the predetermined area is an area which is defined by a maximum point of the first variation information, a minimum point of the second variation information, and a point at which a value of the first variation information is greater than or equal to a value of the second variation information, in the variation in accordance with the lapse of time of the first variation information and the second variation information.

6. The sleep state determination apparatus according to claim 1,
wherein the determination unit determines the sleep state as the REM sleep in a first time zone in which the first extreme value area and the second extreme value area overlap each other, and determines the sleep state as the non-REM sleep in a second time zone in which the first extreme value area and the second extreme value area do not overlap each other.

7. The sleep state determination apparatus according to claim 1,
wherein the determination unit performs smoothing processing and normalization processing on the first variation information and the second variation information.

8. The sleep state determination apparatus according to claim 1, further comprising:
an age information acquisition unit which acquires age information relating to the age,
wherein the determination unit determines a first threshold value and a second threshold value based on the age information, determines the first extreme value area by applying the first threshold value to the first variation information, and determines the second extreme value area by applying the second threshold value to the second variation information.

9. The sleep state determination apparatus according to claim 8,
wherein the determination unit determines the sleep state as the REM sleep in a first time zone in which the first extreme value area and the second extreme value area overlap each other, and determines the sleep state as the non-REM sleep in a second time zone in which the first extreme value area and the second extreme value area do not overlap each other.

10. The sleep state determination apparatus according to claim 8,
wherein the determination unit acquires an incidence ratio of the REM sleep in accordance with the age information, determines the first threshold value such that a first proportion which defines the first extreme value area in the first variation information becomes the incidence ratio, and determines the second threshold value such that a second proportion which defines the second extreme value area in the second variation information becomes the incidence ratio.

11. The sleep state determination apparatus according to claim 2,
wherein the determination unit divides a power spectrum which is obtained through frequency analysis at predetermined frequencies, one of which is regarded as the first index and the other one of which is regarded as the second index.

12. The sleep state determination apparatus according to claim 1,
wherein the measurement unit detects body motion information, and
wherein the determination unit acquires a variation amount indicating a variation of the body motion information and determines whether a subject is in the sleep state or in an awake state, based on the acquired variation amount.

13. The sleep state determination apparatus according to claim 12,
wherein the determination unit calculates a first variance between a first class and a second class, and a second variance within each of the classes, and determines a threshold value, for classifying the variation amount into the first class for determining the awake state and the second class for determining the sleep state, such that the ratio of the first variance and the second variance becomes maximum.

14. A sleep state determination method comprising:
measuring biological information;
calculating a first index in accordance with an activity of a first autonomic nerve and a second index in accordance with an activity of a second autonomic nerve, based on the measured biological information;
deriving first variation information which indicates a variation of the first index and a second variation information which indicates a variation of the second index; and
determining whether a sleep state indicated by the biological information is REM sleep or non-REM sleep, based on the first variation information and the second variation information.

15. A sleep management system in which a sleep state determination apparatus that determines a sleep state and an information processing apparatus are communicably connected to each other,
wherein the sleep state determination apparatus includes
a measurement unit which measures biological information,
a determination unit which calculates a first index in accordance with an activity of a first autonomic nerve and a second index in accordance with an activity of a second autonomic nerve, based on the biological information which is measured by the measurement unit, derives first variation information which indicates a variation of the first index and a second variation information which indicates a variation of the second index, and determines whether a sleep state indicated by the biological information is a REM sleep or a non-REM sleep, based on the first variation information and the second variation information, and
a communication unit which notifies the information processing apparatus of a determination result of the determination unit through communication, and
wherein the information processing apparatus processes information relating to sleep based on the determination result which is notified from the sleep state determination apparatus.
